(19) United States
(12) Patent Application Publication
(10) Pub. No.: US 2026/0219127 A1
(43) Pub. Date: Jul. 30, 2026

(54) SYSTEM AND METHOD FOR PASSIVE OPTICAL MONITORING OF THE CONCENTRATION OF MULTIPLE GASES AND/OR MULTIPLE PARAMETERS AT MULTIPLE POINTS

(71) Applicants: PETRÓLEO BRASILEIRO S.A. - PETROBRÁS, Rio de Janeiro (BR); ALFA TECNOLOGIA E SERVIÇOS EM SENSORIAMENTO DE FIBRA ÓPTICA LTDA, São Bernardo do Campo (BR); FUNDAÇÃO CPQD - CENTRO DE PESQUISA E DESENVOLVIMENTO EM TELECOMUNICAÇÕES, Campinas (BR)

(72) Inventors: Bruno Nogueira AIRES, Campinas (BR); Claudio FLORIDIA, Campinas (BR); Ednardo Moreira SANTOS, Rio de Janeiro (BR); Filipe Henrique CARDOSO, Valinhos (BR); João Batista ROSOLEM, Campinas (BR); João Paulo Vicentini FRACAROLLI, Campinas (BR); João Roberto NOGUEIRA JÚNIOR, Sumaré (BR); Rivael Strobel PENZE, Campinas (BR); Victor DIAGO, São Bernardo do Campo (BR)

(73) Assignees: PETRÓLEO BRASILEIRO S.A. - PETROBRÁS, Rio de Janeiro, RJ (BR); ALFA TECNOLOGIA E SERVIÇOS EM SENSORIAMENTO DE FIBRA ÓPTICA LTDA, São Bernardo do Campo, SP (BR); FUNDAÇÃO CPQD - CENTRO DE PESQUISA E DESENVOLVIMENTO EM TELECOMUNICAÇÕES, Campinas, SP (BR)

(21) Appl. No.: 19/145,653

(22) PCT Filed: Dec. 15, 2023
(86) PCT No.: PCT/BR2023/050454

§ 371 Date: Jul. 3, 2025

(30) Foreign Application Priority Data
Jan. 4, 2023 (BR) 1020230001467

(51) Int. Cl. G01M 3/38

(52) U.S. Cl. CPC G01M 3/38

(57) ABSTRACT

A system and method for passive optical monitoring of the concentration of multiple gases and/or multiple parameters at multiple points for obtaining and monitoring the methane concentration in offshore platforms and onshore structures, and in the fields of mining, landfills and other industrial sectors. The system includes sending, by a remote fiber optic unit, a frequency-modulated light pulse (intra-pulse modulation) through an optical fiber, which is optically selected and directed to optical splitters and delay lines for several measurement points of interest, originating multiple signals that interact with the methane gas in reflective sensors, that is, sensors that reflect light, returning to the remote unit a time signal from N point sensors, which is detected by a single optical receiver and the time signals of which are analyzed individually by calculating the fast Fourier transform (FFT).

### FIELD OF THE INVENTION

This invention belongs to the field of environmental engineering in the application of measuring fugitive methane emissions applied to the oil and gas sector on offshore platforms and onshore structures, as well as in the mining, landfill and other industrial fields where measuring the concentration of methane and other gases at multiple points is necessary for safety and emission control.

### BACKGROUND OF THE INVENTION

The oil and gas industry presents continuous and growing challenges. The greater complexity for the exploration of new fields and the volume of associated data drives the digitization of the sector, aiming to increase the competitiveness of companies, improving the safety of operations and mitigating environmental damage, in addition to allowing them to cope with the continuous fluctuation of prices.

On the other hand, global climate change has been strongly influenced by the emission of greenhouse gases, particularly those associated with the production and use of energy and energy transformation. Among energy sources, fossil fuels, coal, oil and natural gas represent the most significant share of gas emissions globally. Fugitive emissions are leaks in equipment resulting from mechanical operating failures, wear of sealing structures and characteristics inherent to the materials. In natural gas transport systems, these emissions, composed mainly of methane, reduce the energy efficiency of processes and contribute to the intensification of climate change.

Thus, fugitive methane emissions in the production, delivery and use of natural gas can undermine the climate benefit that natural gas has over other fossil fuels, because methane traps heat in the atmosphere much more effectively than carbon dioxide, especially in the short term. In fact, as reported more recently in “Methane Research: The 16 Study Series, Environmental Defense Fund”, the global warming potential of methane in the first 20 years is 84 times more powerful than CO₂. Also, according to this document, methane emissions also represent a waste of natural gas. Annually in the United States, the amount of natural gas lost is enough to power 6 million homes. In addition, the gas lost in the United States in 2016 had the same negative impact on the climate as the annual carbon emissions of 117 million cars, or approximately half of the country's fleet.

Methane is also explosive, fugitive emissions can cause safety problems in addition to the environmental problem. Fugitive methane emissions in the U.S. natural gas industry account for 62.1% of total emissions, with compression stations and delivery stations contributing 21.5% and 10.1% respectively of this total, while combustion and ventilation emissions account for 7.9% and 30% respectively of total methane emissions, as reported in “Estimate of methane emissions from the U.S. natural gas industry, D. A. Kirchgessner, R. A. Lott, R. M. Cowgill, M. R. Harrison, T. M. Shires, Chemosphere, v. 35, n. 6, p. 1365-1390, 1997”.

Currently, on offshore platforms and onshore structures, the monitoring of fugitive emissions is conducted manually on a quarterly or four-monthly basis at tens of thousands of points by a team that must go through the entire installation and conduct the measurement point by point. This procedure is inefficient and makes it impossible to monitor in real time, which would allow the use of computational tools to extract, among other information, trend and behavior analyses and optimization studies, risk and cost reduction in the day-to-day operations of the structures.

Similarly, in sectors such as mining and landfills, continuous monitoring is required to reduce environmental risks and impacts, and to make decisions.

Although especially important, the current monitoring of methane is time-consuming, with a quarterly or four-monthly frequency to obtain the results. Therefore, a system is needed that allows effective monitoring of this gas on platforms, gas processing stations and refineries, as well as in other sectors such as the mining sector and landfills.

### STATE OF THE ART

Some prior art documents describe methods and systems for measuring methane, which will be presented below.

Patent document WO2016075438A1 (Multipoint gas sensing apparatus) describes a modular architecture for a multipoint optical gas detection network. A laser beam from a tunable laser diode source is split and each of the resulting beams is directed via optical fibers to the respective gas detection cells. As the laser beam is configured to sweep a frequency range encompassing a characteristic absorption frequency of a target gas, a measurement of the level of light absorption of the laser beam incident on each cell is correlated with the concentration of the target gas. The photodetectors are arranged to receive the light transmitted through the respective cells and the attenuation detected in the laser beam is attributed to absorption (once other factors have been corrected). The photodetectors are organized into groups in the receiver modules and the receiver modules are provided with dedicated processing capability.

Patent document CN103439289B (Multichannel and multi-point-location gas detection system based on second harmonic detection technique) describes an invention that refers to a multichannel and multipoint gas detection and location system based on a second harmonic detection technique, belonging to the technical field of fiber optic gas detection. According to the system, broadband infrared light sources covering multiple absorption spectrum lines of the same gas or the absorption spectrum lines of multiple gases are used, and an erbium-doped fiber amplified spontaneous emission light source (ASE light source) and a light-emitting diode (LED) are typically selected; the broadband infrared light sources are split into multiple narrowband light sources only respectively covering a certain absorption spectrum line of a given gas, using the wavelength selection characteristics of a Bragg fiber network; and in cooperation with a second harmonic gas detection technique, the system can perform multichannel detection and multipoint location of the concentrations of the same gas or multiple gases. The multi-channel gas detection and multi-point location system allows gas concentrations in multiple channels and point locations to be detected simultaneously using only one light source.

Patent document WO2016189281A1 (Gas sensing apparatus) describes a method for safely detecting and locating leakage locations along the gas pipeline. An optical gas detection system is deployed in the cable duct near the gas pipeline. The concentrations of natural gas present in underground or otherwise enclosed pipelines are measured by a small form factor measurement sensor and, together with the measurements of the linear displacement of the measurement sensor, these concentration measurements can be used to deduce the location of gas leaks.

Patent document CA2301069C (Large scale high speed multiplexed optical fiber sensor network) describes a digital spatial domain and wavelength system for multiplexing fiber Bragg grating sensors comprising: a plurality of optical fibers, each including a plurality of fiber Bragg gratings therein, each fiber Bragg grating having a selective center wavelength that is variable according to the voltage to reflect or transmit light at the corresponding center wavelength according to its voltage; means for providing broadband illumination to the fiber Bragg gratings; means for each optical fiber to carry light to a selected location; and a wavelength dispersing device responsive to the light of each of the wavelength fibers by separating the light in each fiber at the center wavelengths according to the location of each fiber so that the selected location of each fiber and the split wavelength light provides spatially independent signals for each fiber Bragg grating in each optical fiber.

Patent document U.S. Pat. No. 7,688,673B2 (Connector nodes for interrogating optical fiber sensor arrays) describes seismic sensor systems and sensor station topologies, as well as corresponding sensor station cables and components, manufacturing and deployment techniques. For some embodiments, optical ocean bottom seismic station networks are provided, in which sensor stations are efficiently deployed in a modular fashion as a series of array cable modules deployed along a multi-fiber cable.

Patent document WO2000062021A1 (An optical fiber sensor assembly) describes an optical fiber sensor assembly comprising a source of a plurality of substantially monochromatic signals of different frequency; a modulator connected to the output of the source to produce a train of output pulses of the monochromatic signals; a plurality of sensor subassemblies connected to the output of the modulator, each subassembly comprising a drop optical multiplexer, a sensor array comprising a plurality of sensor elements and an add optical multiplexer; a wavelength demultiplexer, having an input coupled to the output of subassemblies; a plurality of detectors, each detector having an input connected to receive a respective output from the wavelength demultiplexer and providing at an output thereof a signal corresponding to a respective frequency of the modulated monochromatic signals; and an interrogation system, having a plurality of inputs connected so that each input receives the output signal from a respective detector.

Patent document U.S. Pat. No. 5,754,293A (Apparatus for the simultaneous acquisition of high bandwidth information in very long arrays containing large numbers of sensor elements) describes an apparatus comprising a source of electromagnetic radiation; a detection network comprising a plurality of detection interferometers, each of which includes a wavelength filter for selecting a band of wavelengths of electromagnetic radiation provided by the source and is suitable for converting the magnitude of one or more physical parameters to a change in a sensor optical path length delay; a reference interferometer for selecting a reference optical path delay; a wavelength selection device for selecting one or more of the wavelength band filtered by one or more of the detection interferometers; and a detector for converting the electromagnetic radiation selected by the wavelength selection device into an electrical signal.

Patent document U.S. Pat. No. 6,212,306B1 (Method and device for time domain demultiplexing of serial fiber Bragg grating sensor arrays) describes an invention that provides a method and device for implementing time division multiplexing of a fiber optic serial Bragg grating sensor array containing more than one Bragg grating. The device provides a pulse reading system that allows a reduction in system noise and an increase in sensor resolution and flexibility. The reflected optical signals from the Bragg grating sensors are blocked by an electronically controlled optical modulator before any wavelength measurement is performed to determine the sensor information.

In the article “Multiplexing of optical fiber gas sensors with a frequency-modulated continuous-wave technique” (H. B. Yu, W. Jin, H. L. Ho, K. C. Chan, C. C. Chan, M. S. Demokan, G. Stewart, B. Culshaw, and Y. B. Liao, Appl. Opt. 40, 1011-1020 (2001)) the use of a frequency-modulated continuous wave technique for multiplexing gas optical fiber sensors is reported. The sensor network is of ladder topology and is interrogated by a tunable laser. The system's performance in terms of detection sensitivity and interference between sensors was investigated and considered limited by the coherent mixing between signals from different channels. The system's performance can be significantly improved by using wavelength modulation-appropriate scanning, along with low pass filtering. The computer simulation shows that an array of 37 acetylene sensors with a detection accuracy of 2,000 parts in 106 for each sensor can be performed. A two-sensor acetylene detection system was experimentally demonstrated with a detection sensitivity of 165 parts in 106 for 2.5 cm gas cells.

In the article “Remote ambient methane monitoring using fiber-optically coupled optical sensor”, (Stephen B. Schoonbaert, David R. Tyner & Matthew R. Johnson, Appl. Phys. B 119, 133-142 (2015)) a tunable diode laser absorption spectroscopy system is described, employing a 2f wavelength modulation spectroscopy measurement scheme. This system was developed for remote monitoring of environmental methane fluctuations by means of fully optical sensors connected by optical fiber. To determine the optimal configuration of the base system, the influence of the frequency and amplitude of the laser drive signal was characterized to achieve a balance between measurement accuracy and system sensitivity. In addition, in relation to the basic system configuration, a 50 and 96% reduction in measurement deviation was achieved through polarization scrambling and thermal stabilization of critical optical components, respectively. The long-term stability of the system and the performance of the system were analyzed using data sets acquired 4 and 12 months after the initial calibration of the system, producing a difference in the accuracy measured within the uncertainties of the calibration gas mixture. Finally, it was determined that the fiber length between individual remote optical sensors can lead to a variable measurement bias, which implies that specific length calibrations for each remote optical sensor may be required for field implementation.

Although there are documents describing methods and systems for obtaining methane concentration through optical fibers, they operate by signal transmission and do not present solutions for the measurement of multiple points that allow their large-scale expansion in a modular way, in addition to these systems using reception with multiple detectors, which increases their complexity and cost. In addition, the methods that make use of frequency analysis fail to compensate for factors that degrade the quality of the optical signals.

These systems also do not present the possibility of concomitant measurement of other physical quantities using the same optical network used for the measurement of methane concentration. Therefore, it would be interesting to develop a system and method capable of solving these problems found in the state of the art, and whose object of the present patent presents and whose advantages are indisputable and described below.

### BRIEF DESCRIPTION OF THE INVENTION

This invention describes a system and method for passive optical monitoring of the concentration of multiple gases and/or multiple parameters at multiple points for obtaining and monitoring the concentration of methane on offshore platforms and onshore structures, as well as in the mining, landfill and other industrial fields where the measurement of methane concentration at multiple points is necessary for safety and emission control. The system consists of the sending, by a remote fiber optic unit, a frequency-modulated light pulse (intra-pulse modulation) in an optical fiber, which is optically selected and directed to optical splitters and delay lines for several measuring points of interest, originating multiple signals that interact with the methane gas in reflective sensors, which are sensors that reflect light, returning to the remote unit a temporal signal of N point sensors, which is detected by a single optical receiver and has its temporal signals analyzed in isolation, calculating the fast Fourier transform (FFT). The FFT signal obtained contains an amplitude A0 at the zero frequency or DC component, f0, an amplitude A1 at the same modulation frequency of the laser, f1, and an amplitude A2 at twice the original frequency of the laser, f2=2×f1. In this way, the system allows obtaining the concentrations (C) of the N sensors from the amplitudes A0, A1 and A2 through the method proposed herein by calculating the average (M) between log (A2/A0) and log (A2/A1), i.e. M=½×[log (A2/A0)+log (A2/A1)], and a calibration curve, where M is invariant with the optical loss of the fiber optic links and depends solely on the concentration, making said system and method independent of the optical attenuation.

This process is repeated for K outputs of an optical switch, positioned in the remote unit, enabling the monitoring of N×K, sensors spread in an industrial plant of interest (for example an offshore platform), serving tens of thousands of points, in a modular and expandable way. The system also allows the concomitant monitoring of other physical quantities by the same optical network through sensors based on FBG (Fiber Bragg Grating).

### BRIEF DESCRIPTION OF THE FIGURES

The figures to which reference is made below are presented to obtain a full and complete visualization of the object of this invention.

FIG. 1 shows a design of the system for detecting fugitive methane emissions, with the split active and passive parts. The active part consists of an interrogator and the passive part consists of a distribution box and the sensor elements.

FIG. 2 shows the operation of the optical signals sending, detecting and analyzing unit that makes up the interrogator element.

FIG. 3A shows the passive part of the system consisting of the distribution box and the optical methane sensing elements.

FIG. 3B shows another embodiment of the passive part of the system that adds common arms and delay lines, which allows greater modularity of the system and the repetition of the use of the distribution box and the optical methane sensing elements, with savings in optical fibers.

FIG. 4 shows the method for obtaining the methane concentration of the return signals, by analyzing the temporal signal and its corresponding Fourier transform.

FIG. 5 shows a way of conducting the optical signals sending, detecting and analyzing unit that makes up the interrogator element that aggregates the use of the FBG interrogator, enabling the concomitant monitoring of other physical quantities.

FIG. 6 shows a way of implementing the passive part of the system consisting of the distribution box and the optical methane sensing elements and which allows the addition of FBG sensing elements for concomitant monitoring of other physical quantities.

### DETAILED DESCRIPTION OF THE INVENTION

This invention describes a system and method for passive optical monitoring of the concentration of multiple gases and/or multiple parameters at multiple points.

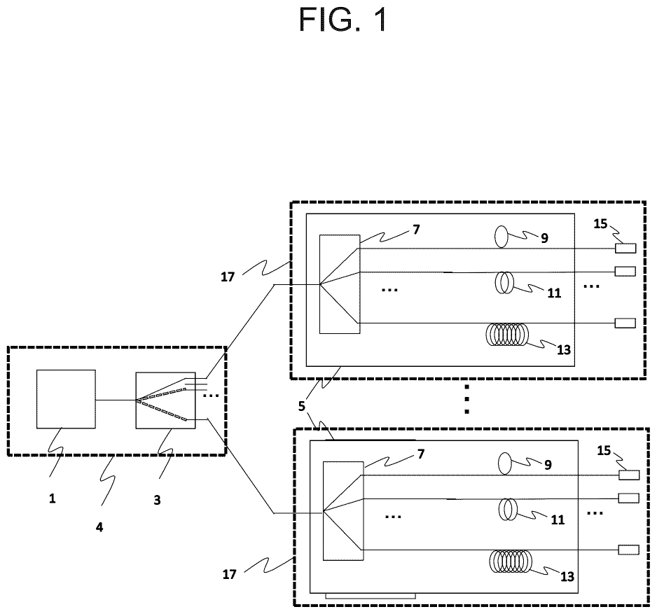

In one of its preferred configurations, as shown in FIG. 1, the system of this invention comprises:
optical signals sending, detecting and analyzing unit (1), wherein the unit emits optical pulses with emission wavelength centered on the methane absorption line 21, with a detector for the detection of the return optical signals and element for the calculation of the methane concentration for each passive sensor element;
optical switch or set of optical switches (3) with the main function of routing the optical signals to K optical arms for the distributing units and respective passive optical sensors;
the optical signals sending, detecting and analyzing unit (1) together with the optical switch or set of optical switches (3) may occupy the same packaging and physical space constituting an element called an optical interrogator (4);
optical splitter (7) consisting of 1 (one) input optical fiber that can be of the single-mode type with N output optical fibers, preferably of the single-mode, bidirectional type, to equally distribute the optical signals to the N sensors;
delay lines (9), (11), . . . , (13) consisting of a fixed and defined length of optical fiber that can be of the single-mode type, related to the duration of the optical pulse, aiming to temporarily delay the optical pulse initially sent from the unit (1) in each arm of the optical splitter (7) so that in the unit (1) it is possible to temporarily split the information coming from the several sensors (15) in the detection process;
the optical splitter elements (7) and the delay lines (9), (11), . . . , (13) can occupy the same packaging and physical space, constituting a distribution box (5);
sensor elements (15) which expand the optical signals in a light beam and allow the interaction of light with the methane gas inside, enabling the quantification of the concentration of this gas, the element must be reflective, the optical signals interacts with the gas and is reflected by a mirror constituting the sensor element and again interacts with the gas, duplicating the optical path of interaction, the optical signals then returns to the element (1);
the optical splitter elements (7) and the delay lines (9), (11), . . . , (13) together with the optical sensors (15) constitute the passive elements (17) of the system and are distributed and repeated in a modular way to increase the number of monitored points, totaling N×K monitoring points.

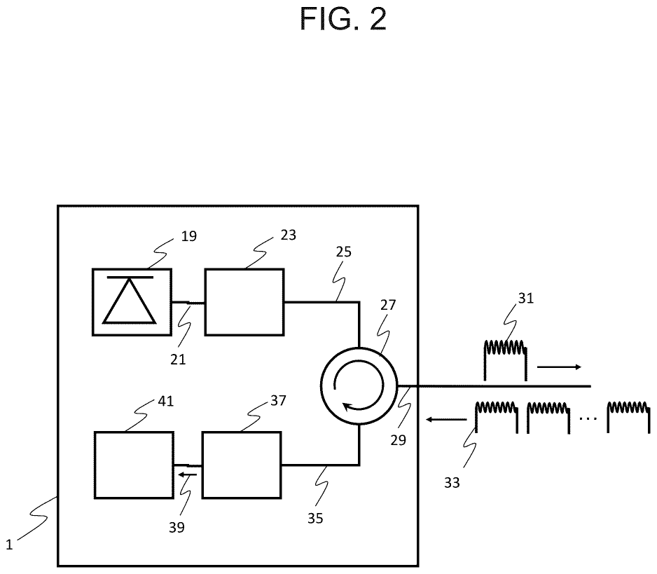

The FIG. 2 shows the details of the sending unit of the system of the invention, comprising the detection and analysis of optical signals (1); in this unit, a laser preferably of the DFB type (19) tuned to the methane λ1 absorption length, for example at 1650.95 nm, has its current modulated by a frequency f1 greater than 1 MHz and sends the optical signals through the optical fiber (21) to an optical device (23) preferably an optical switch with a response time of the order of tens of nanoseconds, or alternatively a semiconductor optical amplifier (SOA), operating in the 1650 nm spectral range. Said device (23) whose main function is to modulate the light in pulses with a defined duration and frequency lower than the light previously modulated in the laser (19), for example 10 kHz, said duration of the light pulse is related to the delay lines (9), (11), . . . , (13), as will be defined later. The produced light pulses are sent to the first optical port (25) of an optical circulator (27) which redirects said pulses through the second optical port (29) of the circulator. The formed optical pulse (31) is sent to passive elements of the system (17), i.e., to the distribution boxes (5) and from there to the respective sensors (15) (shown in FIG. 1) from where multiple pulses (33) return to the second optical port (29) of the optical circulator (27), said pulses being redirected to the third optical port (35) to a single set consisting of a photodetector, an electronic amplifier and a data acquisition board (37), whose output signal (39) is sent to the processing or calculating unit (41). This unit (41) individually analyzes the related signals through the METHOD proposed herein, in each of the optical pulses (33), each pulse containing the delay provided by the delay lines (9), (11), . . . , (13). The methane gas concentrations measured in each of the optical sensors (15) are obtained through the method proposed herein. The processing unit then externalizes the said concentrations obtained.

In another embodiment, the light pulses (31) can be produced directly by the laser (19) without the need for an optical component (23) by means of switching it on or off.

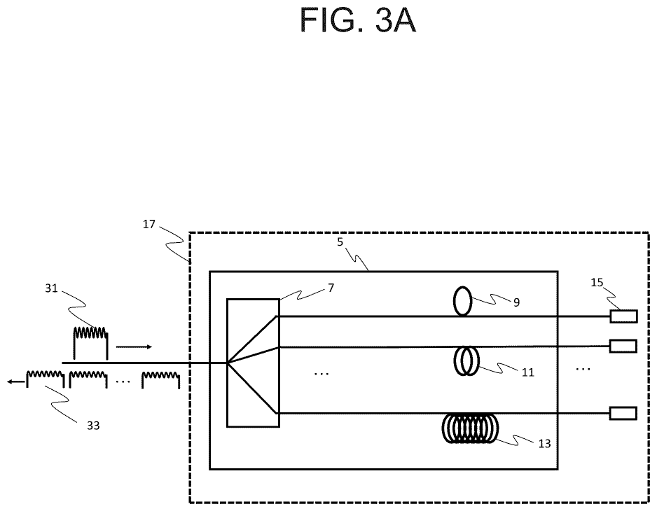

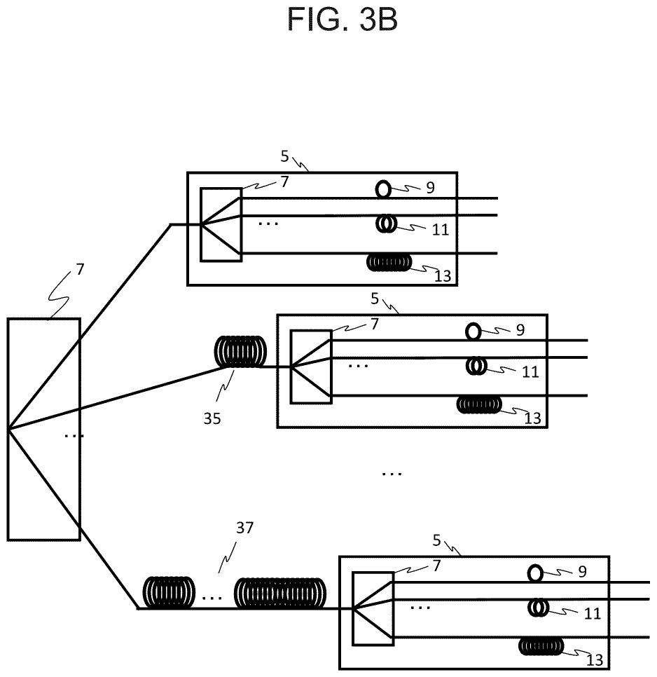

FIG. 3A shows the details of the passive arm (17) of the system of this invention composed of the distribution box (5) and sensors (15). The optical pulse (31) generated by the optical signals sending, detecting and analyzing unit (1) has its optical power split into N optical arms by the optical splitter (7), in the upper arm of said figure the pulse passes through an optical delay line (9) of defined length and related to the width of the pulse sent, so that said optical pulse goes and returns through the optical sensor (15) and passes again through an optical delay line (9) resulting in a known time delay and defined by the delay line (9); similarly in the arm immediately below it, the optical pulse (31) passes on its way to and from the second sensor (15) in the delay line (11), being temporally and spatially delayed to the first, and so on. In the last delay, the pulse passes through a delay line (13) so that it is temporally and spatially delayed to the others. On return to the optical splitter (7) the several pulses are regrouped into a single fiber originating the temporally and spatially spaced return optical pulses (33), the first having passed through the 1st arm, the 2nd through the second, and so on until the Nth pulse coming from the Nth arm. To allow this delay between the return pulses, the delay lines are determined by the width of the pulse sent (31). For example, for a pulse of 1 μs the optical delay lines (9), (11), . . . , (13) must have multiple lengths of 100 m. That is, if the line (9) has a length of 100 m, the delay line (11) must be 200 m and so on until the line (13) which will have a length of N×100 m, for N arms of the optical splitter (7), to avoid overlapping of the return optical pulses (33) of FIG. 2.

FIG. 3B shows a second embodiment of the system of this invention. A first optical division (7) is inserted between the distribution boxes (5) and the several arms of the splitter to provide the delays between the pulses. In each arm, additional delay lines (35), . . . , (37) are inserted with sufficient optical delay lines to temporally and spatially displace the optical pulses of each distribution box. This embodiment represents a saving of the amount of fiber required between the service boxes (5) as it groups elements with common delays. In addition, it allows the system to be installed in a modular way by repeating several identical elements (5) in the passive network.

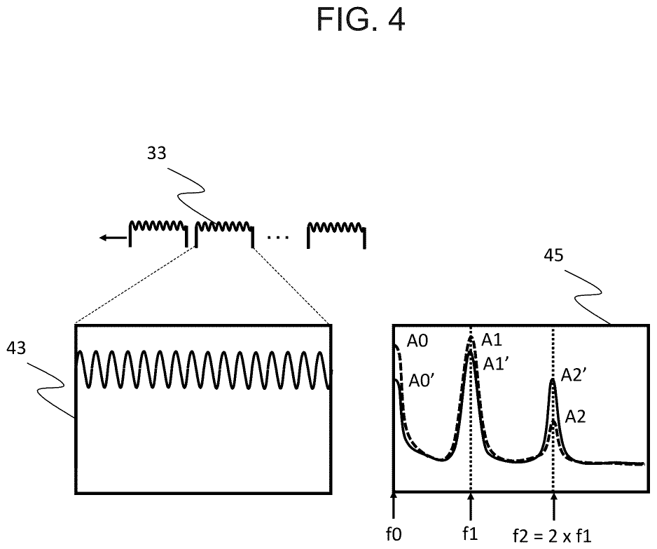

The method for passive optical monitoring of the concentration of multiple gases and/or multiple parameters at multiple points, in one of its preferred configurations, is described below and its understanding is complemented by FIG. 4.

In FIG. 4, the sequence of return pulses (33) detected in the unit (1) is shown. Each of the detected pulses is analyzed individually by observing its temporally modulated signal (intra-pulse modulation) (43) and calculating the Fast Fourier Transform (FFT) of each of the signals with intra-pulse modulation (45). The FFT signal obtained contains an amplitude A0 at frequency zero or DC component, f0, an amplitude A1 at the same modulation frequency as the laser, f1, and an amplitude A2 at twice the frequency, f2=2×f1. The ratio between the amplitude at frequency f2, A2, and the amplitude at frequency f1, A1, i.e., A2/A1 is related, for example, to methane concentration. In general, if there is an increase in concentration, the amplitudes A0, A1 and A2 change (for example, A2 increases to a value A2′). The existence of additional optical loss in the fiber optic link also changes the amplitudes A0, A1 and A2, but this influence of the optical losses in the fiber link is compensated by calculating A2/A0 and A2/A1 in logarithm. The average (M) between log (A2/A0) and log (A2/A1), i.e., M=½×[log (A2/A0)+log (A2/A1)], is invariant with optical loss and depends solely on the gas concentration (C), which is obtained by means of a calibration curve.

Thus, the method of this invention previously described consists of the following steps as described below:
Step 1: capturing the sequence of consecutive optical pulses (33) by, for example, a signal acquisition board (37);
Step 2: separating the several pulses, disregarding the rising and falling edges, obtaining the individual time signals of each sensor (intra-pulse modulation) (43);
Step 3: FFT calculation of each individual time signal of each sensor (45);
Step 4: obtaining the amplitudes at zero frequency or DC component, f0, in the original modulation, f1, and at twice this frequency, f2=2×f1, resulting in amplitudes A0, A1 and A2;
Step 5: calculating the ratio between A2 and A0, A2/A0 and A2 and A1, A2/A1;
Step 6: obtaining the concentration by means of a calibration curve that relates the average (M) of log (A2/A0) and log (A1/A0), i.e., M=½×[log (A2/A0)+log (A2/A1)], and the gas concentration (C); i.e., the calibration curve is the relationship between M and C; the quantity M is invariant with optical loss and depends solely on the gas concentration (C), which is obtained by means of a calibration curve;
Step 7: externalizing the concentration values of the N×K sensors for the user or complementary system;
Step 8: repeat the measurement starting with Step 1.

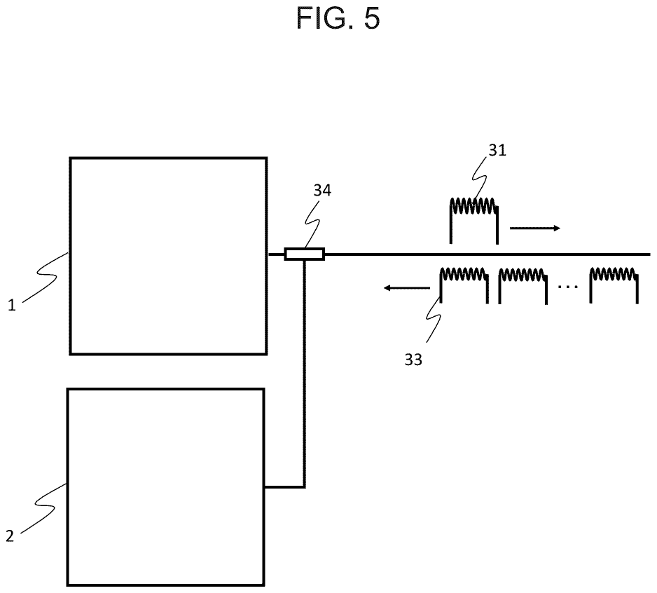

FIG. 5 shows the details of a second embodiment of the optical signals sending, detecting and analyzing unit that incorporates into the system for passive optical monitoring of the concentration of multiple gases and/or multiple parameters at multiple points the functionality of concomitant monitoring of other physical quantities. An FBG interrogator element (2) is added to the optical signals sending, detecting and analyzing unit by means of the wavelength multiplexer device (34) which allows the signal from the FBG interrogator (2) to be sent in the optical band around 1550 nm concomitantly with the modulated signal for optical detection in the specific methane absorption line, for example at 1650.95 nm. When sending the signals to the methane sensors and to the FBG sensors, they are multiplexed by the wavelength multiplexer (34) and can travel through the same fiber to the respective sensors. Upon returning from the respective sensors, the signals in the methane absorption line continue to be detected and analyzed by the optical signals sending, detecting and analyzing unit (1) while the signals from the FBG sensors, which measure other physical quantities, are analyzed by the FBG interrogator element (2) as they are split again by the wavelength multiplexer element (34) which redirects these return signals to the methane and FBG detecting and analyzing units (1) and (2), respectively.

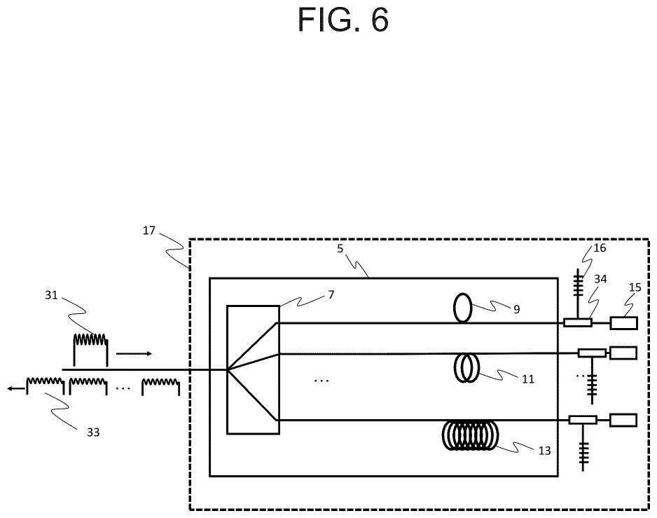

FIG. 6 shows the details of an embodiment of the passive arm (17) composed of the distribution box (5) and methane sensors (15) with the addition of the FBG sensor elements (16) for measuring other physical quantities. Upon arrival of the optical signals, the signals for these FBG sensor elements (16) are split from the signals for the methane sensors (15) by the same wavelength multiplexer element (34). Each signal returns from its respective methane and FBG concentration sensors and is regrouped by the same wavelength multiplexing element (34) traveling the reverse path to the methane optical signals sending, detecting and analyzing unit (1) and FBG interrogation unit (2), as previously explained.

Those skilled in the art will value the knowledge presented herein and may reproduce the invention in the embodiments presented and in other variants, covered in the scope of the attached claims.

1-7. (canceled)

8. A system for passive optical monitoring of the concentration of multiple gases and/or multiple parameters at multiple points, comprising:
an optical signal sending, detecting and analyzing unit, wherein the unit emits optical pulses with emission wavelength centered on the methane absorption line λ1, with a detector for the detection of the return optical signals and element for the calculation of the methane concentration for each passive sensor element;
an optical switch or set of optical switches with a function of routing the optical signals to K optical arms for the distributing units and respective passive optical sensors;
the optical signals sending, detecting and analyzing unit together with the optical switch or set of optical switches occupies the same packaging and physical space constituting an element called an optical interrogator;
an optical splitter comprising one input optical fiber that is a single-mode type with N output optical fibers, to equally distribute the optical signals to the N sensors;
delay lines comprising a fixed and defined length of optical fiber that can be of the single-mode type, related to the duration of the optical pulse, to temporarily delay the optical pulse initially sent from the unit in each arm of the optical splitter so that in the unit it temporarily splits the information coming from the several sensors in the detection process;
the optical splitter elements and the delay lines occupy a same packaging and physical space, constituting a distribution box;
sensor elements which expand the optical signals in a light beam and allow the interaction of light with the methane gas inside, enabling the quantification of the concentration of this gas, the element is reflective, the optical signals interacts with the gas and is reflected by a mirror constituting the sensor element and again interacts with the gas, duplicating the optical path of interaction, the optical signals then returns to the element;
the optical splitter elements and the delay lines, together with the optical sensors, constitute the passive elements (of the system and are distributed and repeated in a modular way to increase the number of monitored points, totaling N×K monitoring points.

9. The system of claim 8, wherein the detection and analysis of optical signals in this unit comprises a laser of the DFB type, tuned to the absorption length of methane λ1, has its current modulated by a frequency f1 greater than 1 MHz and sends the optical signals through the optical fiber to an optical switch with a response time of the order of tens of nanoseconds, or alternatively a semiconductor optical amplifier (SOA), operating in the 1650 nm spectral range; the device modulates the light in pulses with a defined duration and frequency lower than the light previously modulated in the laser, said duration of the light pulse that is related to the delay lines; the light pulses produced are sent to the first optical port of an optical circulator that redirects said pulse through the second optical port of the circulator; the formed optical pulse is sent to passive elements of the system, to the distribution boxes and from there to the respective sensors, from where multiple pulses return to the second optical port of the optical circulator, said pulses being redirected to the third optical port and from this port the pulses are sent to a single set, comprising a photodetector, an electronic amplifier and a data acquisition board, whose output signal is sent to the processing or calculating unit; the unit individually analyzes the related signals in each of the optical pulses, each pulse containing the delay provided by the delay lines; and calculating the methane gas concentrations detected in each of the optical sensors.

10. The system of claim 9, wherein the optical pulse generated by the optical signals sending, detecting and analyzing unit has its optical power split into N optical arms by the optical splitter, in an upper arm, where the pulse passes through an optical delay line of defined length and related to the width of the pulse sent, so that said optical pulse goes and returns through the optical sensor and passes again through the optical delay line resulting in a known time delay defined by the delay line, similarly in the arm immediately below the upper arm, the optical pulse passes on its way to and from the second sensor in the delay line, being temporally and spatially delayed to the first; in a lower arm, the pulse passes through a delay line so that it is temporally and spatially delayed to the others; on the return to the optical splitter the several pulses are regrouped into a single fiber originating the return optical pulses spaced temporally and spatially, the first having passed through a 1st arm, the 2nd through the second, and so on until the Nth pulse coming from the Nth arm; to allow this delay between the return pulses, the delay lines are determined by the width of the pulse sent.

11. The system of claim 10, wherein to provide the delays between the pulses, a first optical division is inserted between the distribution boxes and the several arms of the splitter; in each arm, additional delay lines are inserted with sufficient optical delay lines to temporally and spatially displace the optical pulses of each distribution box.

12. A method developed according to the system of claim 11, comprising:
Step 1: capturing the sequence of consecutive optical pulses by a signal acquisition board;
Step 2: separating the several pulses, disregarding the rising and falling edges, and obtaining the individual time signals of each sensor (intra-pulse modulation);
Step 3: fast Fourier transform (FFT) calculating of each individual time signal of each sensor;
Step 4: obtaining the amplitudes at zero frequency or DC component, f0, in the original modulation, f1, and at twice this frequency, f2=2×f1, resulting in amplitudes A0, A1 and A2;
Step 5: calculating the ratio between A2 and A0, A2/A0 and A2 and A1, A2/A1;
Step 6: obtaining a concentration by a calibration curve that relates the average (M) of log (A2/A0) and log (A1/A0), i.e., M=½×[log (A2/A0)+log (A2/A1)], and a gas concentration; wherein the calibration curve is the relationship between M and C; the quantity M is invariant with optical loss and depends solely on the gas concentration, which is obtained by means of a calibration curve;
Step 7: externalizing the concentration values of the N×K sensors for the user or complementary system;
Step 8: repeating the measurement starting with Step 1.

13. The system of claim 11, wherein the optical signals sending, detecting and analyzing unit incorporates the functionality of concomitant monitoring of other physical quantities; an FBG interrogator element is added to the optical signals sending, detecting and analyzing unit by means of the wavelength multiplexer device that allows the signal from the FBG interrogator to be sent in the optical band around 1550 nm concomitantly with the modulated signal for optical detection in the specific methane absorption line; when sending, the signals to the methane sensors and to the FBG sensors are multiplexed by the wavelength multiplexer, and can travel through the same fiber to the respective sensors; when returning from the respective sensors, the signals in the methane absorption line continue to be detected and analyzed by the optical signals sending, detecting and analyzing unit while the signals from the FBG sensors, which measure other physical quantities, are analyzed by the FBG interrogator element as they are split again by the wavelength multiplexer element that redirects these return signals to the methane and FBG detecting and analyzing units and, respectively.

14. The system of claim 8, further comprising a passive arm composed of the distribution box and methane sensors, with an addition of the FBG sensor elements for measuring other physical quantities; upon arrival of the optical signals, the signals for these FBG sensor elements are split from the signals for the methane sensors by the same wavelength multiplexer element; and each signal returns from its respective methane and FBG concentration sensors, and is regrouped by the same wavelength multiplexer element traveling the reverse path to the methane optical signals sending, detecting and analyzing unit and FBG interrogation unit.
